(19) United States
(12) Patent Application Publication
(10) Pub. No.: US 2025/0222151 A1
(43) Pub. Date: Jul. 10, 2025

(54) DEOFORANT BALL FOR SHOES

(71) Applicant: Hangzhou Cecebear Life Science and Technology Co., Ltd., Hangzhou (CN)

(72) Inventor: Hui Xiong, Hangzhou (CN)

(21) Appl. No.: 19/094,225

(22) Filed: Mar. 28, 2025

(30) Foreign Application Priority Data
Jun. 27, 2024 (CN) 202421492757.2

(51) Int. Cl. A61L 2/23; A61L 9/12

(52) U.S. Cl. CPC A61L 2/23; A61L 9/12; A61L 2202/15; A61L 2202/16; A61L 2209/134

(57) ABSTRACT

The invention provides a deodorant ball for shoes. The deodorant ball for shoes includes a deodorizing ball, a diffuser disk, and a disassembly and assembly unit. The deodorizing ball is provided with two sets and is freely disassembled. The diffuser disk is capable of freely controlling dispersion of deodorizing substances. The disassembly and assembly unit includes a set of disassembly and assembly structures in both inner and outer circles of the diffuser disk. Since the deodorant ball of the present invention are provided with two sets and is freely disassembled and assembled, and the two sets of deodorizing balls are traction-connected through a traction rope. When the application is used to deodorize shoes, the deodorizing balls can be separated and placed in their respective shoe cavities to realize the deodorizing operation of a pair of shoes.

### FIELD OF THE INVENTION

The invention relates to the technical field of cleaning device for shoes, in particular, to a deodorant ball for shoes.

### BACKGROUND OF THE INVENTION

The causes of shoe odor in existing technologies mainly include the following aspects:

Excessive sweating of feet. The sweat glands on the feet are more dense than other parts of the body, so they tend to sweat easily. When your feet sweat, the moisture, salts, fats, proteins and other components in the sweat provide a rich source of nutrition for bacteria, promoting their large-scale reproduction inside shoes, thus producing unpleasant odors.

Shoes are not breathable. If shoes have non-breathable materials or a strong closed design, it will cause poor air circulation inside the shoes, rising humidity and temperature, providing favorable conditions for bacterial growth. In this way, bacteria multiply in large numbers inside the shoes and decompose substances in sweat, producing unpleasant odors.

Bacteria thrive in warm, moist environments like those found inside shoes. Bacteria that break down organic matter in sweat are especially prevalent. These bacteria produce a variety of chemicals during their growth process, some of which have unpleasant odors.

Therefore, in the prior art, some deodorizing articles for shoes are designed to deodorize shoes (including some silver ion sprays, some deodorizing bags, etc.). These deodorizing articles are usually placed inside the shoes or on the shoe racks to eliminate the odor in the shoes by releasing deodorants or absorbing moisture. Deodorant balls for shoes mainly use activated carbon, bamboo charcoal, minerals and other adsorption materials to absorb moisture, odor and bacteria in shoes. At the same time, some products will also add ingredients such as plant extracts or spices to provide additional deodorizing and refreshing effects.

However, when using silver ion spray to deodorize, it is expensive and easy to cause respiratory discomfort and disease during use. When using a deodorant bag, the time of use is not long enough, resulting in poor deodorization effect.

### SUMMARY OF THE INVENTION

The purpose of the invention is to provide a deodorant ball for shoes, in order to solve the above-mentioned shortcomings in the background technology.

The purpose of the invention is to provide a deodorant ball for shoes, in order to solve the above-mentioned background technology.

In order to achieve the above purpose, a technical scheme is provided by the present invention. A deodorant ball for shoes, wherein the deodorant ball for shoes is provided with two sets of deodorizing ball and is freely disassembled and assembled; a diffuser disk, wherein the diffuser disk is capable of freely controlling the dispersion of deodorizing substances; and a disassembly and assembly unit, wherein the disassembly and assembly unit include a set of disassembly and assembly structures in both inner and outer circles of the diffuser disk.

As a further scheme of the present invention: an separation plate is fixedly installed inside the deodorizing ball to separate the deodorizing ball into a plurality of cavities, one side of the deodorizing ball is movably connected to a first diffuser disk or a second diffuser disk,, diffuser ports inside the first diffuser disk or the second diffuser disk match a number and a position of the cavities, one side of the first diffuser disk is rotatably connected to a movable disk via a rotating shaft, and the movable disk via a rotating shaft, and the movable disk controls the connection or closing of the cavities and the diffuser ports.

As a further scheme of the present invention: the deodorant ball for shoes also includes a matching buckle and a matching seat. The matching buckle includes a buckle base, a spring sheet and a step. The buckle base is fixedly connected with the first diffuser disk. The spring sheet is fixedly connected with the buckle base, the step is embedded in the outer periphery of the shrapnel, the matching seat includes a seat hole and a seat ring, the seat hole is fixedly connected to the second diffuser disk, the seat ring is fixedly connected to the seat hole, and the step is movable connected to the seat ring.

As a further embodiment of the invention, one side of the first diffuser disk is fixedly connected with a first sealing ring and one side of the second diffuser disk is fixedly connected with a second sealing ring. The outer periphery of the first sealing ring is movably connected to the inner periphery of the second sealing ring.

As a further embodiment of the invention, one side of the second sealing ring is fixedly connected with a second magnet, and one side of the first diffuser disk is fixedly connected with a first magnet. The end of the first sealing ring away from the first diffuser disk is in contact with the second diffuser disk, and the end of the second magnet away from the second sealing ring is magnetically attracted to the first magnet.

As a further embodiment of the invention, a flush hole is formed in the inner part of the first diffuser disk or second diffuser disk. One end of the rotating shaft is fixedly connected with a pointer disk and the pointer disk is movable within the flush hole.

As a further embodiment of the invention, it also includes a traction rope. The two sets of odor-removing balls are connected by the traction rope.

Compared with the existing technology, the beneficial effects of the invention are:
1. Since there are two sets of deodorizing balls that can be freely disassembled and assembled, and the two sets of deodorizing balls are connected by traction ropes, when using the present application to deodorize shoes, the deodorizing balls can be separated and placed in their respective shoe cavities to realize deodorization of a pair of shoes. After deodorization is completed, the deodorizing balls can be taken out of the shoe cavity by traction ropes, and then the two sets can be merged by using disassembly and assembly unit, complete the storage of the deodorant ball and ensure that the deodorant ball will not be lost.
2. By placing activated carbon and different scents in four cavities separated by the separation plate, it can achieve the effects of deodorization, moisture removal and flavor enhancement inside the shoe cavity. At the same time, through setting an activity disk that is connected to the first divergent disk or second divergent disk, anyone opening can be selected for odor dispersion, then three other sets of divergent openings are covered up, which can improve the effective use period of the equipment and reduce the frequency of replacing internal activated carbon and fragrance.
3. The dual sealing of the deodorizing ball can be realized by using the inner and outer rings, which effectively ensures the service life of the deodorizing ball.

### BRIEF DESCRIPTION OF THE DRAWINGS

By reading the detailed description of the preferred embodiments below, various other advantages and benefits will become clear to those of ordinary skill in the art. The drawings are only used for the purpose of illustrating the preferred embodiments and are not considered as a limitation to the invention. Also, throughout the drawings, the same reference numerals are used to denote the same components. In the drawings:

FIG. 1 is a closed view of a deodorant ball in one embodiment of the present invention;

FIG. 2 is an unfolded view of the deodorant ball in the present invention;

FIG. 3 is a schematic diagram of the separation structure of the first diffuser disk and the deodorant ball in an embodiment of the present invention;

FIG. 4 is a schematic diagram of the separation structure of the second diffuser disk and the deodorant ball in an embodiment of the present invention;

FIG. 5 is a schematic diagram of the separation structure of the first diffuser disk or second diffuser disk in the present invention;

FIG. 6 is a schematic diagram of the structure of the matching buckle in an embodiment of the present invention; and

FIG. 7 is a structural schematic diagram of the matching seat in an embodiment of the present invention.

### DESCRIPTION OF THE PREFERRED EMBODIMENTS

In order to make the purpose and advantages of the invention clearer, the invention will be further described below in conjunction with the embodiments. It should be understood that the specific embodiments described here are only used to explain the invention and are not used to limit the invention.

It should be understood that in the description of the invention, orientations or position relationships indicated by terms upper, lower, front, back, left, right, inside, outside and the like are orientations or position relationships are based on the direction or position relationship shown in the drawings, which is only for ease of description, rather than indicating or implying that the device or element must have a specific orientation, be constructed and operated in a specific orientation, and therefore cannot be understood as a limitation of the invention.

Further, it should also be noted that in the description of the invention, terms “mounting”, “connected” and “connection” should be understood broadly, for example, may be fixed connection and also may be detachable connection or integral connection; may be mechanical connection and also may be electrical connection; and may be direct connection, also may be indirection connection through an intermediary, and also may be communication of interiors of two components. Those skilled in the art may understand the specific meaning of terms in the invention according to specific circumstance.

In order to explain the technical solutions of the present invention more clearly, specific embodiments are used for description below.

### Embodiments

In the embodiment of the present invention, a deodorant ball for shoes includes a deodorizing ball 1, a traction rope 2, and a disassembly and assembly unit. The deodorizing ball 1 is provided with two sets and is freely disassembled and assembled. The two sets of deodorizing balls 1 are connected by traction. The inside of the deodorizing ball 1 is fixedly installed with an separation plate 3 that divides the deodorant ball 1 into several cavities, one side of the deodorizing ball 1 is connected with a first diffuser disk 4 or a second diffuser disk 5, the number and position of the diffuser ports inside the first diffuser disk 4 or the second diffuser disk 5 match the number and position of the cavities, the side of the first diffuser disk 4 is connected with a movable disc 10 through a rotating shaft 11, and the movable disc 10 controls the communication or closure of the cavity and the diffuser ports, the disassembly and assembly unit include a set disassembly structure of each set of the inner and outer rings of the diffuser disk.

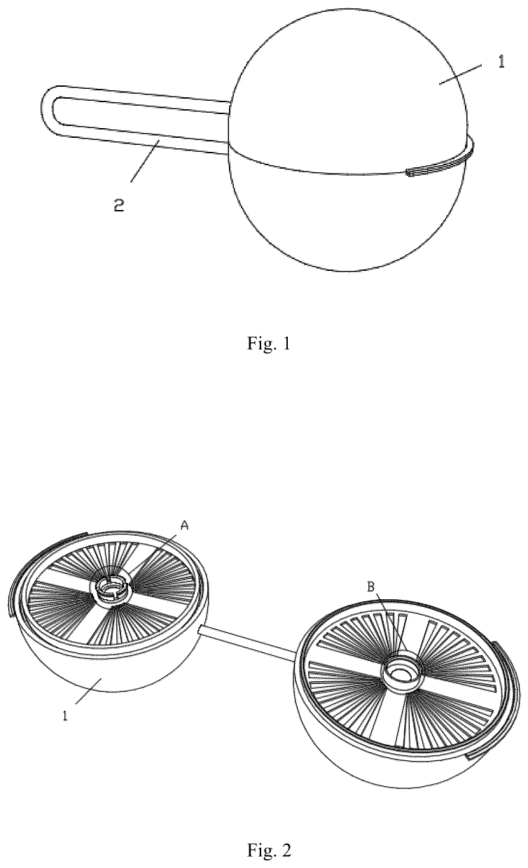

Other embodiments of the invention: Please refer to FIG. 1 and FIG. 2. Since there are two sets of deodorizing bells 1 that can be freely disassembled and assembled, and the two sets of deodorizing bells 1 are connected by a traction rope 2, when using the present application for shoe deodorization, the deodorizing bell 1 can be separated and placed in each shoe cavity to achieve the deodorizing operation of a pair of shoes.

After deodorization, the deodorizing ball 1 can be taken out from the shoe cavity through the traction rope 2. Then two sets of deodorizing balls 1 are combined by using disassembly and assembly unit to complete storage of the deodorizing ball 1 and ensure that the deodorizing ball 1 will not be lost.

As a further scheme of the present invention: including a diffuser disk, the diffuser disk can freely control the divergence of the deodorizing substance, the inside of the deodorizing ball 1 is fixedly installed with an separation plate 3 that divides the deodorizing ball 1 into several cavities, and one side of the deodorizing ball 1 is connected with a first diffuser disk 4 or a second diffuser disk 5, the number and position of the diffuser ports in the first diffuser disk 4 or the second diffuser disk 5 are matched with the number and position of the cavities, the movable disk 10 is rotatably connected to one side of the first diffuser disk 4 through the rotating shaft 11, and the movable disk 10 controls the communication or closure of the cavities and the diffuser ports.

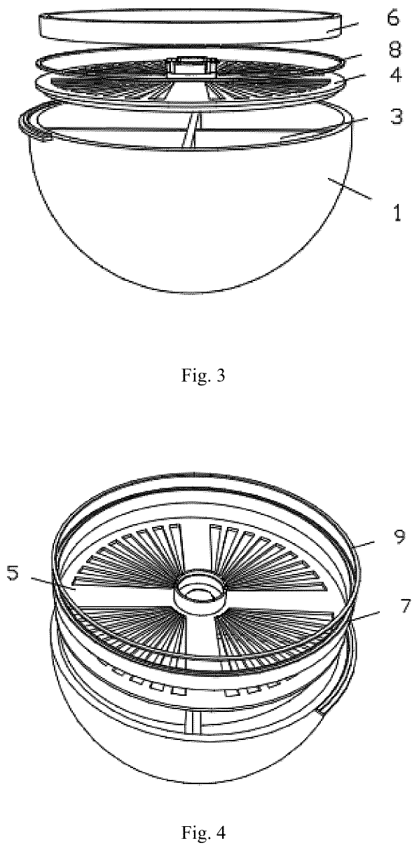

Other embodiments of the present invention: Please refer to FIG. 3, FIG. 4 and FIG. 5, by putting activated carbon fragrances with different flavors in the four cavities separated by the separation plate 3, the effects of deodorization, moisture removal and odor enhancement in the shoe cavity can be realized. At the same time, the movable disc 10 is provided and the movable disc 10 is connected with the first diffuser disk 4 or the second diffuser disk 5, you can choose any opening for deodorization and divergence, and then cover the other three sets of divergence openings with all your heart, which can improve the validity period of the equipment and reduce the replacement frequency of internal activated carbon and fragrance.

As a further embodiment of the invention, the disassembly and assembly unit include a set of disassembly structures on both inner and outer circles of the diffuser disk. The disassembly and assembly unit for the inner circle include matching buckle 14 and matching seat 15, while those for the outer circle include first sealing ring 6 and second sealing ring 7.

Inner ring: the matching buckle 14 includes a buckle base 141, spring sheet 142 and step 143. The buckle base 141 is fixedly connected to the first diffuser disk 4, and the spring sheet 142 includes a plurality of retractable elastic structure and is fixedly connected to the buckle base 141, the step 143 is embedded in the outer periphery of the shrapnel 142, the matching seat 15 includes a seat hole 151 and a seat ring 152, the seat hole 151 is fixedly connected with the second diffuser disk 5, the seat ring 152 is fixedly connected with the seat hole 151, and the step 143 is movable with the seat ring 152.

Outer ring: the first diffuser disk 4 is fixedly connected to a first sealing ring 6 on one side, and the second diffuser disk 5 is fixedly connected to a second sealing ring 7 on another side. The outer periphery of the first sealing ring 6 is movably connected with the inner periphery of the second sealing ring 7.

Other embodiments of the present invention: Please refer to FIG. 3 and FIG. 4. On the one hand, the outer periphery of the first sealing ring 6 is movably connected with the inner periphery of the second sealing ring 7, so that the two sealing rings composed of the first sealing ring 6 and the second sealing ring 7 can block the deodorizing ball 1, and at the same time, the connection can also ensure that the fragrance will not escape from the deodorizing ball 1 after the two deodorizing balls 1 are merged, thus ensuring the service life.

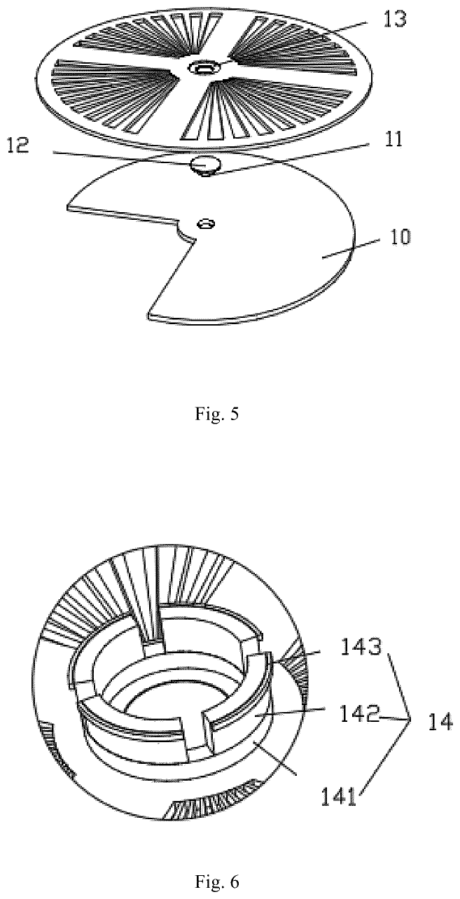

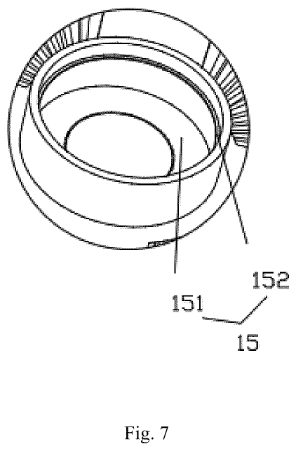

Other embodiments of the invention: Please refer to FIG. 6 and FIG. 7. On the other hand, the retractable spring sheet 142 is inserted into the seat hole 151 after being moved, and the spring sheet 142 is relaxed and offset to make the step 143 clamped with the seat ring 152, thereby realizing the connection between the inner ring of the deodorizing ball 1. As a further embodiment of the invention, one side of the second sealing ring 7 is fixedly connected with a second magnet 9. One side of the first diffuser disk 4 is fixedly connected with a first magnet 8. The end of the first sealing ring 6 away from the first diffuser disk 4 is in contact with the second diffuser disk 5. One end of the second magnet 9 away from the second sealing ring 7 is magnetically attracted to the first magnet 8.

Other embodiments of the present invention: Please refer to FIG. 3 and FIG. 4. By means of magnetic attraction connection between first magnet 8 and second magnet 9, the closure strength of first sealing ring 6 and second sealing ring 7 can be strengthened, further avoiding fragrance leakage.

As a further embodiment of the invention, a flush hole 13 is formed in the inner part of the first diffuser disk 4 or second diffuser disk 5. One end of the rotating shaft 11 is fixedly connected with a pointer disk 12, and the pointer disk 12 is movable within the flush hole 13.

Other embodiments of the invention: Please refer to FIG. 5, the position of the opening of the movable disk 10 can be adjusted by pressing and rotating the pointer disk 12 with the thumb, so as to select the fragrance output, which is easy to operate.

The working principle of the present invention is: because two sets of deodorizing balls 1 are provided and is freely disassembled and assembled, and the two sets of deodorizing balls 1 are connected by traction rope 2, when using the present application to deodorize shoes, the deodorizing balls 1 can be separated and placed in their respective shoe cavities to realize the deodorant operation on a pair of shoes. After Deodorization is completed, the deodorizing balls 1 can be taken out from the shoe cavity by traction rope 2, then, the two sets of deodorizing balls 1 are combined by using the disassembly and assembly unit to complete the storage of the deodorizing balls 1 and ensure that the deodorizing balls 1 will not be lost.

In addition, by placing activated carbon fragrances with different flavors in the four cavities separated by the separation plate 3, the effects of deodorization, moisture removal and odor enhancement in the shoe cavity can be realized. At the same time, by setting the movable plate 10 and connecting the movable plate 10 with the first Diffuser disk 4 or the second Diffuser disk 5, any opening can be selected for deodorization and divergence, and the other three diffuser openings can be fully shielded by dirt, it can improve the validity period of the equipment and reduce the replacement frequency of internal activated carbon and fragrance.

So far, the technical solution of the invention has been described in conjunction with the preferred embodiments shown in the drawings. However, it is easily understood by those skilled in the art that the protection scope of the invention is obviously not limited to these specific embodiments. Without departing from the principle of the invention, those skilled in the art can make equivalent changes or substitutions to the relevant technical features, which will fall into the protection scope of the invention.

The above are only preferred embodiments of the invention rather than limits to the invention. Those skilled in the art may make various modifications and changes to the invention. Any modification, equivalent replacement, improvement and the like made within the spirit and principle of the invention all should be included in the protection scope of the invention.

What is claimed is:

1. A deodorant ball for shoes, comprising:
a deodorizing ball, the deodorizing ball is provided with two sets and is freely disassembled and assembled,
a diffuser disk, wherein the diffuser disk is capable of freely controlling dispersion of deodorizing substances; and
a disassembly and assembly unit, wherein the disassembly and assembly unit includes a set of disassembly and assembly structures in both inner and outer circles of the diffuser disk.

2. The deodorant ball for shoes according to claim 1, wherein an separation plate is fixedly installed inside the deodorizing ball to separate the deodorizing ball into a plurality of cavities, one side of the deodorizing ball is movably connected to a first diffuser disk or a second diffuser disk, diffuser ports inside the first diffuser disk or the second diffuser disk match a number and a position of the cavities, one side of the first diffuser disk is rotatably connected to a movable disk via a rotating shaft, and the movable disk via a rotating shaft, and the movable disk controls the connection or closing of the cavities and the diffuser ports.

3. The deodorant ball for shoes according to claim 2, further comprising:
a matching buckle and a matching seat, the matching buckle includes a buckle base, a spring sheet and a step, the buckle base is fixedly connected to the first diffuser disk, the spring sheet comprises a plurality of retractable elastic structures and is fixedly connected to the buckle base, the step is embedded in the outer periphery of the spring sheet, the matching seat includes a seat hole and a seat ring, the seat hole is fixedly connected to the second diffuser disk, the seat ring is fixedly connected to the seat hole, and the step is movably connected to the seat ring.

4. The deodorant ball for shoes according to claim 3, wherein one side of the first diffuser disk is fixedly connected with a first sealing ring and one side of the second diffuser disk is fixedly connected with a second sealing ring, and an outer periphery of the first sealing ring is movably connected with an inner periphery of the second sealing ring.

5. The deodorant ball for shoes according to claim 4, wherein one side of the second sealing ring is fixedly connected with a second magnet, and one side of the first diffuser disk is fixed connected with a first magnet; one end of the first sealing ring away from the first diffuser disk is in contact with a second diffuser disk, and one end of the second magnet away from the second sealing ring is magnetically connected to a first magnet.

6. The deodorant ball for shoes according to claim 5, wherein a flush hole is provided inside the first diffuser disk or the second diffuser disk, one end of the rotating shaft is fixedly connected to a pointer disk, and the pointer disk is movably connected to the flush hole.

7. The deodorant ball for shoes according to claim 6, further comprising:
a traction rope, wherein two sets of the deodorizing balls are connected to a traction rope.
